(19) United States
(12) Patent Application Publication
(10) Pub. No.: US 2025/0241520 A1
(43) Pub. Date: Jul. 31, 2025

(54) ELEVATORS FOR MEDICAL DEVICES AND RELATED METHODS

(71) Applicants: Boston Scientific Scimed, Inc., Maple Grove, MN (US); Boston Scientific Medical Device Limited, Galway (IE)

(72) Inventors: Aditya DHANOTIYA, Indore (IN); Pooja Bhuvanesh KULKARNI, Pune (IN); Shrikant Vasant RAUT, Pune (IN); James WELDON, Newton, MA (US)

(73) Assignees: Boston Scientific Scimed, Inc., Maple Grove, MN (US); Boston Scientific Medical Device Limited, Galway (IE)

(21) Appl. No.: 19/036,474

(22) Filed: Jan. 24, 2025

Related U.S. Application Data
(60) Provisional application No. 63/624,833, filed on Jan. 25, 2024.

(51) Int. Cl. A61B 1/00

(52) U.S. Cl. CPC A61B 1/00147; A61B 1/00098

(57) ABSTRACT

Elevators for medical devices and related methods are described, including an elevator having a first side wall, a second side wall, and a guide surface for contacting an accessory device. The guide surface may extend between the first side wall and the second side wall. A distal portion of the second side wall may extend further upward in a direction perpendicular to a longitudinal axis of the elevator than a distal portion of the first side wall.

### CROSS-REFERENCE TO RELATED APPLICATIONS

This application claims the benefit of priority to U.S. Provisional Application No. 63/624,833, filed on Jan. 25, 2024, which is incorporated by reference herein in its entirety.

### TECHNICAL FIELD

Various aspects of this disclosure relate generally to elevators of medical devices and related methods. In particular, aspects of this disclosure relate to elevators of medical devices adapted for accessing a target site, e.g., the pancreas or biliary tract, during an endoscopic procedure, among other aspects.

### BACKGROUND

During a medical procedure, such as Endoscopic Retrograde Cholangiopancreatography (ERCP), an operator may position an endoscope adjacent to a major duodenal papilla of a patient, and extend one or more medical instruments through a working channel of the endoscope. An elevator may be disposed at a distal end of the endoscope and may be actuatable in order to change an orientation of the one or more medical instruments to extend the one or more medical instruments into the major duodenal papilla.

### SUMMARY

Each of the aspects disclosed herein may include one or more aspects of the features described in connection with any of the other disclosed aspects.

According to some aspects of the present disclosure, an elevator for a medical device may include a first side wall, a second side wall, and a guide surface for contacting an accessory device. The guide surface may extend between the first side wall and the second side wall. A distal portion of the second side wall may extend further upward in a direction perpendicular to a longitudinal axis of the elevator than a distal portion of the first side wall.

According to some aspects, the distal portion of the second side wall may be angled inward towards the first side wall. In some examples, the guide surface may include a first side portion at least partially on the first side wall and a second side portion at least partially on the second side wall. The second side portion may extend further upward in the direction perpendicular to the longitudinal axis than the first side portion. In some examples, the distal portion of the second side wall may include a surface that faces at least partially in a proximal direction. In some examples, a distalmost end of the distal portion of the second side wall may be adjacent a central portion of the guide surface. The central portion may be between the first side portion and the second side portion. In some examples, the first side portion of the guide surface may be connected to a first side surface of the first side wall by a first side edge. The second side portion of the guide surface may be connected to a second side surface of the second side wall by a second side edge. A height of the second side edge at the distal portion of the second side wall may be greater than a height of the first side edge. In some examples, the distal portion of the second side wall may form a closed end of the second side portion of the guide surface. The second side surface of the second side wall may include a first portion and a second portion. The second portion may extend to a distalmost edge of the elevator and may be angled inward relative to the first portion towards the first side wall. In some examples, an edge of the second portion may extend approximately vertically from the distalmost edge of the elevator. In some examples, a portion of the edge of the second portion may be axially aligned with the distalmost edge of the elevator. In some examples, the edge of the second portion may have a second portion that curves away from a central longitudinal axis of the guide surface. In some examples, the second side edge may include an upward slope extending towards a distal end of the elevator. In some examples, a height of the first side edge and a height of a proximal portion of the second side edge may be the same. In some examples, a guide surface may include a planar side portion at the distal portion of the second side wall. In some examples, the first side wall and the second side wall may extend to a planar distal-facing surface, which may have a “U” shape.

According to some aspects of the present disclosure, an elevator for a medical device may include a first side wall and a second side wall. A proximal portion of the second side wall may be substantially parallel to an entirety of the first side wall. A distal portion of the second side wall may curve toward the first side wall. In some examples, a surface of the distal portion of the second side wall may face at least partially in a proximal direction. In some examples, the elevator may include a guide surface that may extend from the first side wall to the second side wall. The guide surface may include a first portion adjacent to the first side wall, a second portion adjacent to the second side wall, and a central portion between the first portion and the second portion. The first portion and the central portion of the guide surface may have open distal ends. The second portion of the guide surface may have a closed distal end.

According to some aspects of the present disclosure, an elevator for a medical device may include a first side wall, a second side wall, and a guide surface for contacting an accessory device. The guide surface may extend between the first side wall and the second side wall. The guide surface may include a first portion adjacent to the first side wall, a second portion adjacent to the second side wall, and a central portion between the first portion and the second portion. In some examples, distal ends of the first portion and the central portion of the guide surface may be open. The second side wall may form a closed distal end of the second portion. In some examples, a surface of the second side wall may face at least partially in a proximal direction.

### BRIEF DESCRIPTION OF THE DRAWINGS

The accompanying drawings, which are incorporated in and constitute a part of this application, illustrate exemplary aspects of this disclosure and together with the description, serve to explain the principles of the present disclosure.

FIGS. 1A and 1B illustrate an exemplary medical device, in accordance with some aspects of this disclosure.

FIG. 2 illustrates an exemplary method of using the exemplary medical device of FIGS. 1A and 1B, in accordance with some aspects of this disclosure.

FIGS. 3A-3D illustrate features of an exemplary elevator for use with the exemplary medical device of FIGS. 1A and 1B, in accordance with some aspects of this disclosure.

FIG. 4 illustrates features of another exemplary elevator for use with the exemplary medical device of FIGS. 1A and 1B, in accordance with some aspects of this disclosure.

FIGS. 5A-5C illustrate features of a further exemplary elevator for use with the exemplary medical device of FIGS. 1A and 1B, in accordance with some aspects of this disclosure.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of +10% of the stated numeric value or range.

Although duodenoscopes are referenced herein for illustration purposes, it will be appreciated that the disclosure encompasses any suitable medical device configured to allow an operator to access and view internal body anatomy of a subject (e.g., patient) and/or to deliver medical instruments or accessory devices, such as, for example, biopsy forceps, graspers, baskets, snares, probes, scissors, retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, endoscopes, bronchoscopes, gastroscopes, endoscopic ultrasonography (“EUS”) scopes, colonoscopes, ureteroscopes, laparoscopes, cystoscopes, aspiration scopes, sheaths, catheters, or any other suitable delivery device or medical device may be used in connection with the features described herein.

Features of the medical devices herein may improve accessibility to various target sites within a patient's body, e.g., a major duodenal papilla, a bile duct, a pancreatic duct, etc., among other aspects. Inability to gain access to the major duodenal papilla, the bile duct, and/or the pancreatic duct or repeated attempts to do so may result in post-ERCP complications, such as pancreatitis. According to some aspects of the present disclosure, the medical device may include an elevator to, e.g., change an orientation of one or more medical instruments. According to some aspects of the present disclosure, the elevator may include side portions of varying heights and/or shapes to facilitate angling or orienting one or more medical instruments towards a desired location. In some aspects, the elevator may include curved and/or smooth surfaces that may allow for one or more medical instruments to move and/or glide more easily along surfaces of the elevator.

In some exemplary medical procedures, e.g., ERCP, an operator may access the bile duct or the pancreatic duct of a patient by positioning the medical device adjacent to the major duodenal papilla. With respect to a distal end of the medical device and/or a view shown on a display and provided by an imager of the medical device, the opening to the bile duct may typically be located in an upper left quadrant of the major duodenal papilla, e.g., between 10 o'clock and 1 o'clock, and the opening to the pancreatic duct may typically be located on a right half quadrant of the major duodenal papilla, e.g., between 1 o'clock and 4 o'clock. In some aspects, the elevator may facilitate angling or orienting one or more medical instruments towards the bile duct. In some aspects, the elevator may facilitate angling or orienting one or more medical instruments towards the pancreatic duct.

It will be appreciated that reference to any particular procedure is provided only for convenience and not intended to limit the disclosure. A person of ordinary skill in the art would recognize that the concepts underlying the disclosed devices and application methods may be utilized in any suitable procedure, medical or otherwise. Reference will now be made in detail to examples of the present disclosure described above and illustrated in the accompanying drawings. Wherever possible, the same reference numbers will be used throughout the drawings to refer to the same or like parts.

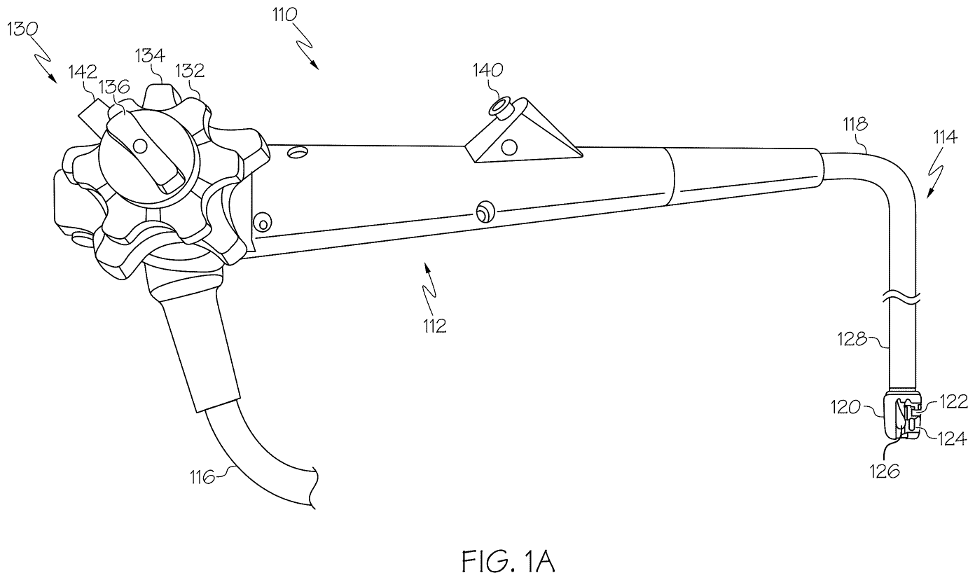

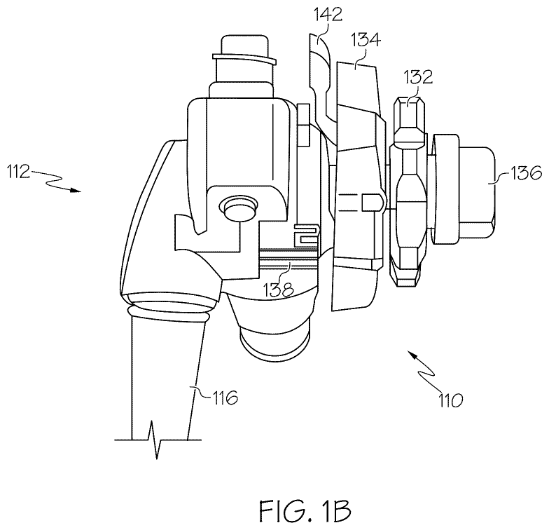

FIG. 1A depicts an exemplary medical device 110 (e.g., a duodenoscope) having a handle 112 and an insertion portion 114. FIG. 1B shows a proximal end of handle 112. Medical device 110 may also include an umbilicus 116 for purposes of connecting medical device 110 to sources of, e.g., air, water, suction, power, etc., as well as to image processing and/or viewing equipment. Insertion portion 114 may include a sheath or shaft 118 and a distal tip 120. Distal tip 120 may include an imaging device 122 (e.g., a camera) and a lighting source 124 (e.g., an LED or an optical fiber). In some examples, as shown in FIG. 1A, distal tip 120 may be side-facing (i.e., side viewing). That is, imaging device 122 and lighting source 124 may face radially outward, perpendicularly, approximately perpendicularly, or otherwise transverse to a longitudinal axis of shaft 118 and distal tip 120.

Distal tip 120 may also include an elevator 126 for changing an orientation of a medical instrument inserted in a working channel of medical device 110. Elevator 126 may alternatively be referred to as a swing stand, pivot stand, raising base, or any suitable other term. Elevator 126 may be rotatable via, e.g., an actuation wire or another control element that extends from handle 112, through shaft 118, to elevator 126.

A distal portion of shaft 118 that is connected to distal tip 120 may have a steerable section 128. Steerable section 128 may be, e.g., an articulation joint. Shaft 118 and steerable section 128 may include a variety of structures which are known or may become known in the art.

Handle 112 may have one or more actuators/control mechanisms 130. One or more of control mechanisms 130 may provide control over steerable section 128. One or more of control mechanisms may allow for provision of air, water, suction, etc. For example, handle 112 may include control knobs 132, 134 for left, right, up, and/or down control of steerable section 128. For example, one of knobs 132, 134 may provide left/right control of steerable section 128, and the other of knobs 132, 134 may provide up/down control of steerable section 128. Handle 112 may further include a first locking mechanism 136 and a second locking mechanism 142 (e.g., knobs or levers) for preventing steering and/or braking of steerable section 28 in at least one of an up, down, left, or right direction. Handle 112 may include a control lever 138 (see FIG. 1B). Control lever 138 may raise and/or lower elevator 126, via connection between control lever 138 and an actuating wire that extends from control lever 138, through shaft 118, to elevator 126. A port 140 may allow passage of a medical instrument through port 140, into a working channel of the medical device 110, through shaft 118, to distal tip 120.

Although FIG. 1A depicts a side-viewing medical device 110 (a device having imaging device 122 and/or lighting source 124 facing substantially radially outward), it will be appreciated that medical device 110 may alternatively be a forward-viewing device (a device having imaging device 122 and/or lighting source 124 facing substantially distally). In aspects, medical device 110 may include both a side-viewing imaging device 122 and/or lighting source 124 and a forward-facing imaging device 122 and/or lighting source 124.

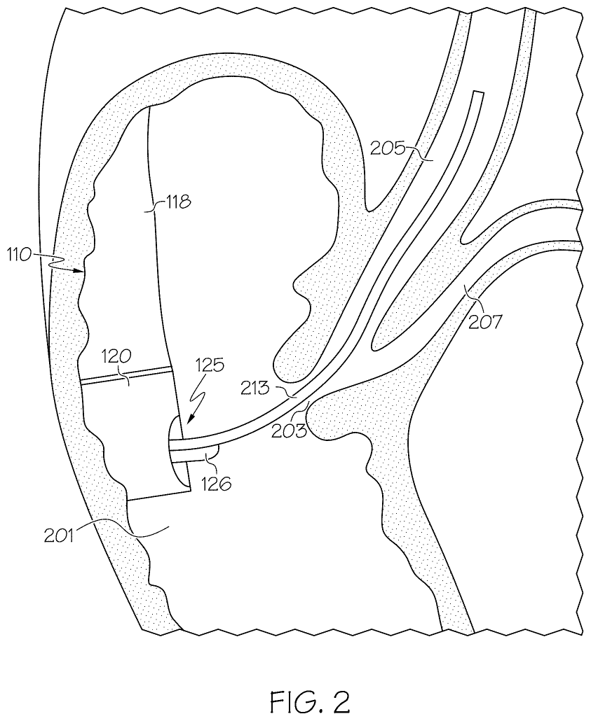

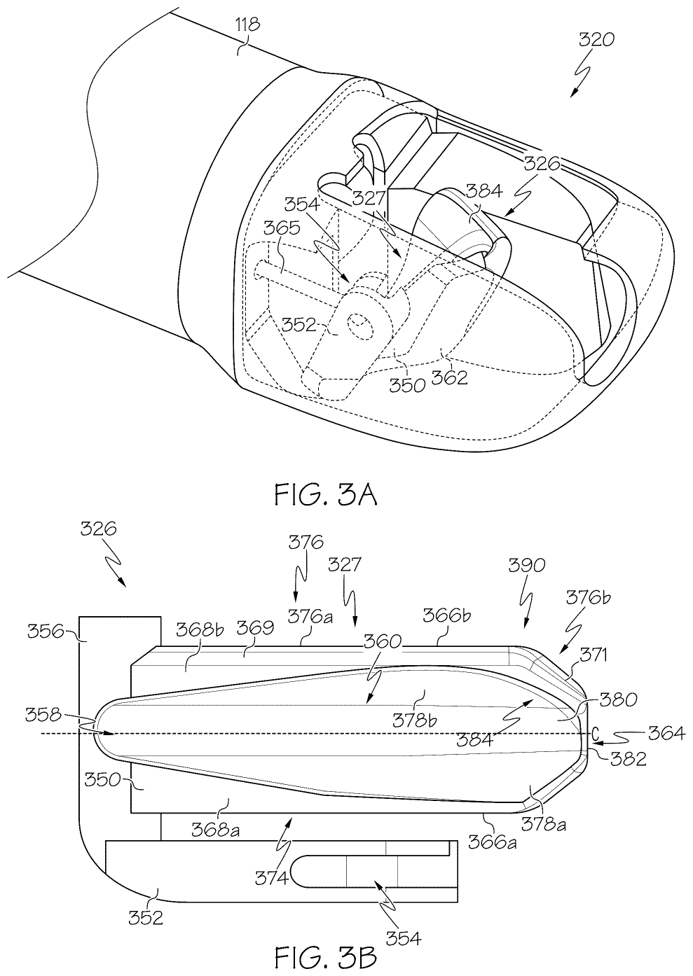

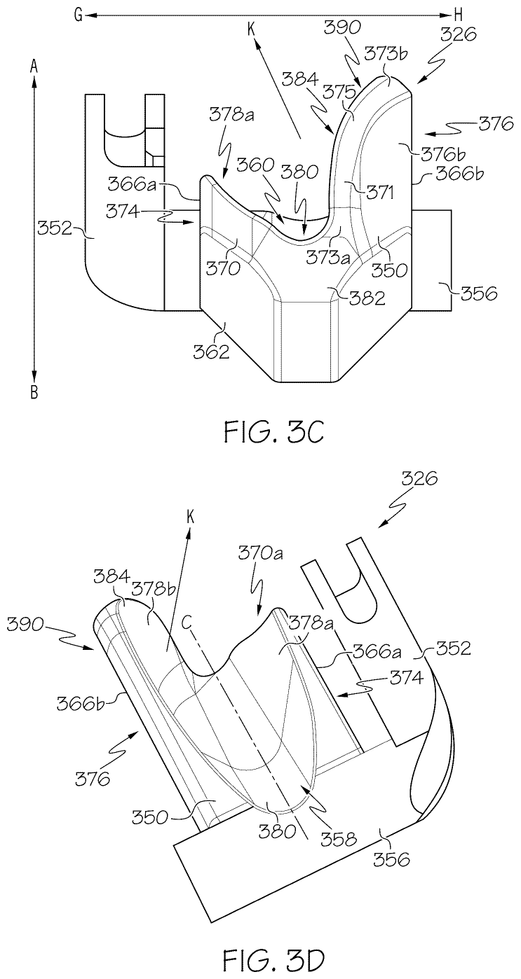

FIG. 2 illustrates shaft 118 of medical device 110 once it has been advanced through a duodenum 201 of a patient and positioned adjacent to a major duodenal papilla 203. An operator may insert a medical instrument and/or an accessory device 213 (e.g., a tome, a knife, a cannula, a guidewire, or any other type of medical instrument) into port 140, and pass medical instrument 213 through shaft 118 via a working channel 125 to distal tip 120. Medical instrument 213 may exit working channel 125 at distal tip 120. The operator may use control lever 138 to raise elevator 126 and angle medical instrument 213 toward major duodenal papilla 203. The operator may cannulate the papilla using medical instrument 213 in order to obtain access to a bile duct 205 or a pancreatic duct 207. The operator may extend medical instrument 213 into bile duct 205 or pancreatic duct 207 to facilitate a medical procedure. In FIG. 2, medical instrument 213 is shown extended into common bile duct 205. As mentioned above, the elevators described herein may facilitate angling or orienting one or more medical instruments 213 towards the bile duct or towards the pancreatic duct.

FIGS. 3A-3D illustrate features of an exemplary elevator 326 that may include any of the features of elevator 126, unless otherwise specified herein. Features of elevator 326 may facilitate angling or orienting one or more medical instruments in a direction K (shown in FIGS. 3C and 3D) and/or in an upward direction towards a target site, e.g., the bile duct, among other aspects. In FIG. 3A, elevator 326 is shown within a housing of an exemplary distal tip 320 that may be used in combination with shaft 118 of medical device 110 (e.g., in place of distal tip 120). Distal tip 320 may include any of the features of distal tip 120, unless otherwise specified herein.

Elevator 326 may include a body 350 and an arm 352. Arm 352 may include a coupler 354 (e.g., an opening, slot, and/or other structure) for coupling a control wire 365 to arm 352. As control wire 365 is moved or urged distally or proximally, elevator 326 may rotate about an axle 356 (shown in FIGS. 3B-3D). For example, as shown in FIG. 3A, moving control wire 365 proximally raises elevator 326 away from a bottom surface of the housing of distal tip 320. Axle 356 may be at a proximal end of elevator 326 (e.g., a proximalmost end of elevator 326), coupled to a proximal end 358 of body 350. Proximal end 358 of body 350 may be opposite a distal end 364 of body 350.

Body 350 may include a guide surface 360, which may be configured to contact and change an orientation of a medical instrument. Guide surface 360 may be on a front side of body 350. Guide surface 360 may extend between proximal end 358 and distal end 364 of body 350, and between first and second side walls 366a, 366b. As disclosed below, guide surface 360 may also extend on an inner surface of second side wall 366b. Guide surface 360 may have a smooth profile to allow for a medical instrument to move and/or glide more easily along guide surface 360. Body 350 may further include a back surface 362. Back surface 362 may be on an opposite side of body 350 from guide surface 360. When elevator 326 is in a lowered configuration, back surface 362 may face downward, toward a bottom of the housing of distal tip 320. When elevator 326 is in a lowered configuration, guide surface 360 may face upward/radially outward, in a generally same direction as that faced by imaging device 122 and lighting source 124.

As shown particularly in FIG. 3C, body 350 may include first side wall 366a (closer to arm 352) including a first side surface 374, and second side wall 366b (farther from arm 352) including a second side surface 376. First side surface 374 may be an outer surface of first side wall 366a, and second side surface 376 may be an outer surface of second side wall 366b. First side wall 366a may be on an opposite side of body 350 from second side wall 366b. First and second side surfaces 374, 376 may extend from guide surface 360 to back surface 362.

As shown particularly in FIG. 3C, at least a portion of second side wall 366b may extend further upward than first side wall 366a in a direction A. Direction A/an upward direction may be a direction perpendicular to a central longitudinal axis C of body 350. Direction A may be a direction of a vector extending from back surface 362 toward guide surface 360. Direction A may be opposite a direction B. Direction B may be a direction of a vector extending from guide surface 360 to back surface 362. In other words, at least a portion of second side wall 366b may extend higher than first side wall 366a.

In some examples, first side wall 366a and second side wall 366b may have a substantially same height from proximal end 358 to an intermediate portion 327 of body 350. Intermediate portion 327 may be between proximal end 358 and distal end 364. For example, a portion of second side wall 366b between proximal end 358 and distal end 364 may be substantially parallel to an entirety of first side wall 366a. Distally of intermediate portion 327, second side wall 366b may taper upwardly in direction A, such that a height of second side wall 366b may be greater than a height of first side wall 366a from intermediate portion 327 to distal end 364.

Guide surface 360 may include a first side portion 378a, a second side portion 378b, and a central portion 380 between first and second side portions 378a, 378b. A central distal-facing surface 382 may extend downward (in direction B) from a distalmost end of central portion 380.

At least a portion of first side portion 378a may be on first side wall 366a. At least a portion of second side portion 378b may be on second side wall 366b. Due to the greater height of second side wall 366b as compared to first side wall 366a, second side portion 378b may have a greater height than first side portion 378a. A distalmost end of first side portion 378a may taper distally toward a distalmost end of central portion 380. A distalmost end of central portion 380 may be approximately straight and may extend between distalmost ends of first side portion 378a and second side portion 378b.

As shown in FIGS. 3C and 3D, a distal end portion 390 of second side wall 366b may be curved or angled inward towards first side wall 366a. For example, a distalmost end of distal end portion 390 may be adjacent to central portion 380. Distal end portion 390 may wrap inward toward central longitudinal axis C. An inner surface 384 of distal end portion 390 may correspondingly curve inward towards first side wall 366a. Thus, distal end portion 390 may block or distally enclose a distalmost end of second side portion 378b of guide surface 360. Inner surface 384 may face at least partially in a proximal direction. In other words, a normal line extending from inner surface 384 may face at least partially in a proximal direction.

A proximal portion of second side wall 366b (a portion proximal of distal end portion 390) may be approximately parallel to central longitudinal axis C and to first side wall 366a. As particularly shown in FIG. 3C, whereas first side portion 378a of guide surface 360 may be open on its distalmost end, second side portion 378b of guide surface 360 may have a closed distalmost end because distal end portion 390 may form a closed distal surface.

Second side surface 376 may include a first portion 376a and a second portion 376b. Second portion 376b may be distal to first portion 376a. Second portion 376b may be disposed on distal end portion 390 of second side wall 366b. Second portion 376b may be angled inward relative to first portion 376a towards first side wall 366a, in accordance with the inward wrapping of second side wall 366b.

As shown in FIGS. 3B and 3D, first side surface 374 of first side wall 366a may be connected to first side portion 378a of guide surface 360 by a first edge 368a. As used herein, the term “edge” includes surfaces that have a non-zero width. Second side surface 376 of second side wall 366b may be connected to second side portion 378b of guide surface 360 and/or inner surface 384 by a second edge 368b.

Second edge 368b may have a first portion 369 and a second portion 371. First portion 369 may extend from proximal end 358 to distal end portion 390 of second side wall 366b. Proximally of intermediate portion 327, first portion 369 may be approximately parallel to central longitudinal axis C. Distally of intermediate portion 327, first portion 369 may extend axially and taper upward in direction A. Second portion 371 may curve inward towards first side wall 366a. A shape of second portion 371 will be described in further detail below. Second edge 368b may have a varying thickness (i.e., in G/H directions perpendicular to A/B directions, and perpendicular to central longitudinal axis C) from the proximal end to the distal end of second edge 368b.

First edge 368a may extend approximately parallel to central longitudinal axis C. A portion of second edge 368b proximal of intermediate portion 327 may have a same height along direction A as first edge 368a. In other words, a proximal portion of second edge 368b may be level with first edge 368a. Portions of second edge 368b distal of intermediate portion 327 may have a greater height than first edge 368a.

As shown in FIG. 3C, second portion 371 of second edge 368b may extend between second portion 376b of second side surface 376 and inner surface 384. Second portion 371 may have a first end 373a that is adjacent to/contacting central distal-facing surface 382 (which is a distalmost edge of elevator 326). Second portion 371 may have a second end 373b that is adjacent to/contacting first portion 369. From first end 373a, second portion 371 may extend in a generally upward direction, e.g., approximately vertically, to an inflection point 375. For example, first end 373a may be axially aligned with central distal-facing surface 382. From inflection point 375 to second end 373b, second portion 371 may curve/flare in direction H, toward first portion 369. Overall, second portion 371 may extend more in an up/down (A/B) direction than in a lateral (G/H) direction. A first side distal edge 370 may extend from first edge 368a to central distal-facing surface 382. First side distal edge 370 may extend between first side portion 378a and back surface 362. From a distal end of first edge 368a, first side distal edge 370 extends distally and inwardly, toward central longitudinal axis C. First side distal edge 370 may be approximately straight. A normal line extending from first side distal edge 370 may extend distally and toward direction G (distally and away from second side wall 366b).

First edge 368a and first portion 369 of second edge 368b may extend approximately the same amount in a distal direction. Second portion 371 of second edge 368b may extend distally of a distalmost end of first edge 368b. Second portion 371 of second edge 368b and first side distal edge 370 may extend a same distal amount.

During an exemplary medical procedure, shaft 118 and distal tip 320, including elevator 326, may be positioned adjacent a target site, e.g., major duodenal papilla 203. An operator may extend a medical instrument, e.g., medical instrument 213, through a working a channel of shaft 118 and out of an opening of the working channel at distal tip 320, such that medical instrument 213 contacts and/or rests on guide surface 360 of elevator 326, e.g., at central portion 380. The operator may engage control lever 138 to move control wire 365 proximally and raise elevator 326 away from the bottom surface of the housing of distal tip 320. Second side wall 366b (e.g., second side portion 378b of guide surface 360) may urge medical instrument 213 towards direction K and/or in an upward direction. During a medical procedure such as, e.g., ERCP, direction K and/or an upward direction may be a direction towards the bile duct. In other words, second side portion 378b may exert a force on medical instrument 213 in a direction K and/or towards the bile duct.

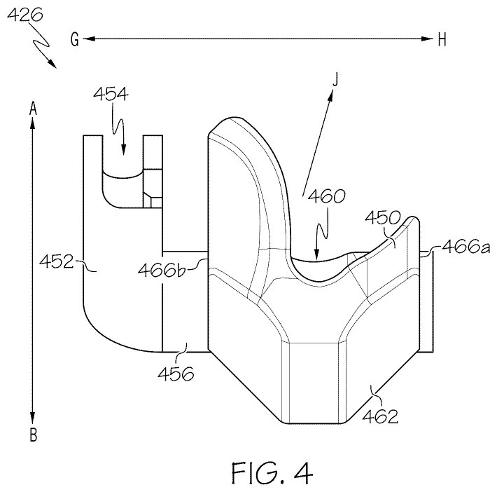

FIG. 4 illustrates features of another exemplary elevator 426 that may include any of the features of elevators 126, 326, unless otherwise specified herein. Elevator 426 may be used in combination with distal tips 120, 320 (e.g., in place of elevators 126, 326). Similar to elevator 326, elevator 426 may include an arm 452 including a coupler 454, and an axle 456. Elevator 426 may include a body 450 including any of the features of body 350.

Body 450 may include a first side wall 466a, which may have any of the features discussed above for first side wall 366a. Body 450 may include a second side wall 466b having any of the features discussed above for second side wall 366b. However, whereas first side wall 366a may be on a side of body 350 that is closer to arm 352, first side wall 466a may be on a side of body 450 that is further from arm 452. And whereas second side wall 366b may be on a side of body 350 that is further from arm 352, second side wall 466b may be on a side of body 450 that is closer to arm 452.

Body 450 may include a guide surface 460 on an opposite side of body 450 from a back surface 462. Guide surface 460 may have any of the features of guide surface 360, and back surface 462 may have any of the features of back surface 362. Features of body 450, including guide surface 460 may be a mirror image of features of body 350, including guide surface 360, about a plane extending in A/B directions along a central longitudinal axis of the respective body 350, 450.

In this example, features of elevator 426 may facilitate angling or orienting one or more medical instruments 213 in a direction J (i.e., in a direction opposite of direction K shown in FIGS. 3A-3D) and/or an upward direction towards a target site, e.g., the pancreatic duct, among other aspects. For example, surface(s) of second wall 466b may urge a medical instrument 213 in direction J and/or in an upward direction.

Elevator 426 may be used in an exemplary medical procedure similar to the exemplary medical procedure described above for FIGS. 3A-3D and in place of elevator 326. In this example, the operator may extend or move medical instrument 213 along guide surface 460. When elevator 426 is raised, surfaces of second side wall 466b or other portions of body 450 that correspond to the surfaces described above for elevator 326 may urge medical instrument 213 towards direction J and/or in an upward direction. During a medical procedure, e.g., ERCP, elevator 426 may exert a force on instrument 213 towards the pancreatic duct.

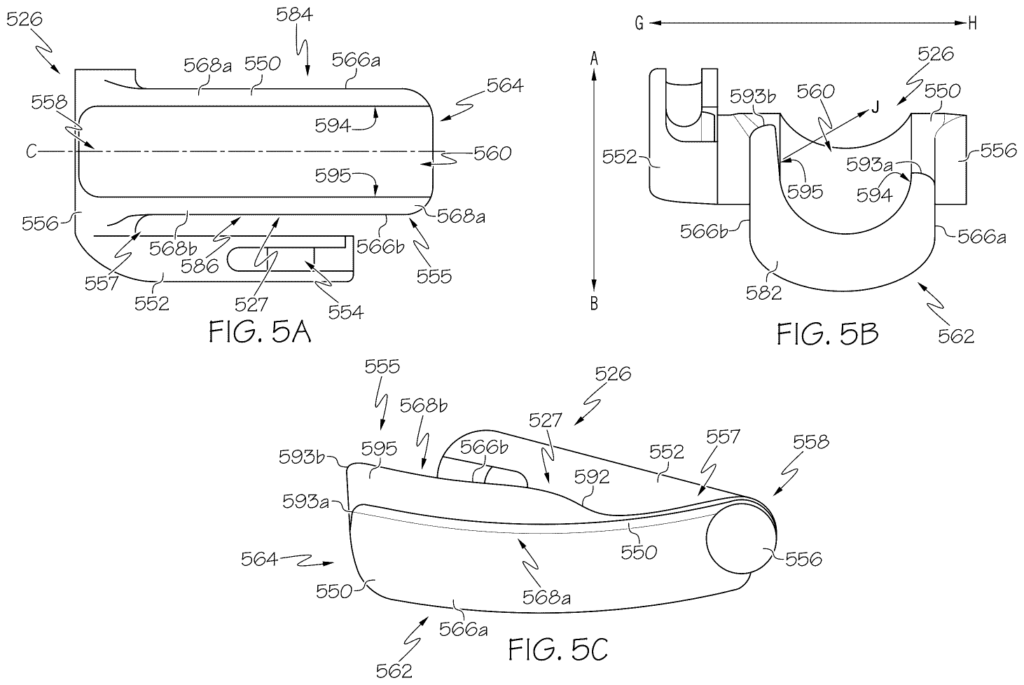

FIGS. 5A-5C illustrate features of another exemplary elevator 526 that may include any of the features of elevators 126, 326, 426, unless otherwise specified herein. Elevator 526 may be used in place of elevators 126, 326 in distal tips 120, 320. Similarly to elevators 326, 426, elevator 526 may include an arm 552 including a coupler 554, and an axle 556. Features of elevator 526 may facilitate angling or orienting one or more medical instruments in direction J and/or an upward direction towards a target site, e.g., the pancreatic duct, among other aspects. Elevator 526 may have a greater length (i.e., in a direction parallel to a longitudinal axis of elevator 526) than elevators 126, 326, 426, and may include a guide surface 560 covering a greater surface area of elevator 526 compared to guide surfaces 360, 460 of elevators 326, 426, which may accommodate larger medical instruments. In other words, guide surface 560 may be deeper and/or wider than guide surfaces 360, 460.

Guide surface 560 may be on a front side of a body 550 of elevator 526. Guide surface 560 may extend between a proximal end 558 and a distal end 564 of body 550, and between first and second side walls 566a, 566b. Guide surface 560 may have a smooth profile to allow for a medical instrument to move and/or glide more easily along guide surface 560. Body 550 may further include a back surface 562. Back surface 562 may be on an opposite side of body 550 from guide surface 560. When elevator 526 is in a lowered configuration, back surface 562 may face downward, toward a bottom of a housing of a distal tip, e.g., distal tips 120, 320. When elevator 526 is in a lowered configuration, guide surface 560 may face upward/radially outward, in a generally same direction as that faced by imaging device 122 and lighting source 124.

Body 550 may have a curved shape, e.g., body 550 may have a substantially C or U cross-sectional shape, as shown particularly in FIG. 5B. Body 550 may include a first side wall 566a (farther from arm 552) and a second side wall 566b (closer to arm 552). As shown in FIG. 5C, second side wall 566b may have a distal portion 555 that is distal of an intermediate portion 527 of body 550, and a proximal portion 557 that is proximal of intermediate portion 527 of body 550. Second side wall 566b may extend further upward than first side wall 566a in direction A (defined above). In other words, distal portion 555 of second side wall 566b may extend higher than first side wall 566a in direction A. Proximal portion 557 of second side wall 566b may have a substantially same height as first side wall 566a. First and second side walls 566a, 566b may extend to a distal-facing surface 582 at distal end 564. Distal-facing surface 582 may be substantially planar and may extend between guide surface 560 and back surface 562. Distal-facing surface 582 may have a substantially “C” or “U” shape.

Guide surface 560 may include a first side portion 594 on first side wall 566a. Guide surface 560 may include a second side portion 595 on second side wall 566b. Due to the greater height of second side wall 566b as compared to first side wall 566a, second side portion 595 may have a greater height than first side portion 594. As shown in FIG. 5A, a first side surface 584 of first side wall 566a may be connected to first side portion 594 by a first side edge 568a, and a second side surface 586 of second side wall 566b may connected to second side portion 595 by a second side edge 568b. Portions of second side portion 595 of guide surface 560 may be substantially planar, e.g., portions adjacent to second side edge 568b.

First and second side edges 568a, 568b may have a substantially same thickness in G/H directions from proximal end 558 to distal end 564 of body 550. First side edge 568a may extend parallel to central longitudinal axis C of body 550. Second side edge 568b may extend approximately parallel to central longitudinal axis C of body 550 along proximal portion 557 of second side wall 566b, and proximal portion 557 may have a same height as first side edge 568a. As shown particularly in FIG. 5C, distal portion 555 of second side edge 568b may include an upward slope 592. For example, second side edge 568b may slope upwards (further along direction A) moving towards distal end 564, such that distal portion 555 may have a greater height than first side edge 568a. As shown in FIGS. 5B and 5C, distal end 593b of second side edge 568b is positioned above distal end 593a of first side edge 568a. Distal-facing surface 582 may extend from first distal end 593a of first side edge 568a to second distal end 593b of second side edge 568b.

Elevator 526 may be used in an exemplary medical procedure similar to the exemplary medical procedure described above for FIG. 4 and in place of elevator 426. In this example, the operator may extend or move medical instrument 213 along guide surface 560. When elevator 526 is raised, second side wall 566b (e.g., second side portion 595 of guide surface 560) may urge medical instrument 213 towards direction J and/or an upward direction. During a medical procedure such as, e.g., ERCP, elevator 526 may exert a force on instrument 213 towards the pancreatic duct.

Alternatively, first side wall 566a of body 550 may have similar features discussed above for second side wall 566b, and second side wall 566b may have similar features discussed above for first side wall 566a. In this alternative, features of elevator 526 may facilitate angling or orienting one or more medical instruments in direction K (i.e., in a direction opposite of direction J) and/or in an upward direction, towards a target site, e.g., the bile duct, among other aspects.

Depending on a type of medical procedure, an operator may select a distal tip, e.g., distal tip 120 or 320, including an elevator, e.g., elevator 126, 326, 426, or 526, and connect the selected distal tip to a shaft, e.g., shaft 118, and a handle, e.g., handle 112. For example, during an ERCP procedure, an operator may select elevator 326 to access a bile duct, or the operator may select elevator 426 or 526 to access a pancreatic duct.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. An elevator for a medical device, comprising:
a first side wall;
a second side wall; and
a guide surface for contacting an accessory device,
wherein the guide surface extends between the first side wall and the second side wall, and
wherein a distal portion of the second side wall extends further upward in a direction perpendicular to a longitudinal axis of the elevator than a distal portion of the first side wall.

2. The elevator of claim 1, wherein the distal portion of the second side wall is angled inward towards the first side wall.

3. The elevator of claim 1, wherein the guide surface includes a first side portion at least partially on the first side wall and a second side portion at least partially on the second side wall, wherein the second side portion extends further upward in the direction perpendicular to the longitudinal axis than the first side portion.

4. The elevator of claim 3, wherein the distal portion of the second side wall includes a surface that faces at least partially in a proximal direction.

5. The elevator of claim 3, wherein a distalmost end of the distal portion of the second side wall is adjacent a central portion of the guide surface, wherein the central portion is between the first side portion and the second side portion.

6. The elevator of claim 3, wherein the first side portion of the guide surface is connected to a first side surface of the first side wall by a first side edge, wherein the second side portion of the guide surface is connected to a second side surface of the second side wall by a second side edge, and wherein a height of the second side edge at the distal portion of the second side wall is greater than a height of the first side edge.

7. The elevator of claim 6, wherein the distal portion of the second side wall forms a closed end of the second side portion of the guide surface.

8. The elevator of claim 7, wherein the second side surface of the second side wall includes a first portion and a second portion, and wherein the second portion extends to a distalmost edge of the elevator and is angled inward relative to the first portion towards the first side wall.

9. The elevator of claim 8, wherein an edge of the second portion extends approximately vertically from the distalmost edge of the elevator.

10. The elevator of claim 9, wherein a portion of the edge of the second portion is axially aligned with the distalmost edge of the elevator.

11. The elevator of claim 8, wherein the edge of the second portion has a second portion that curves away from a central longitudinal axis of the guide surface.

12. The elevator of claim 6, wherein the second side edge includes an upward slope extending towards a distal end of the elevator.

13. The elevator of claim 12, wherein a height of the first side edge and a height of a proximal portion of the second side edge are the same.

14. The elevator of claim 1, wherein the guide surface includes a planar side portion at the distal portion of the second side wall.

15. The elevator of claim 1, wherein the first side wall and the second side wall extend to a planar distal-facing surface, wherein the distal-facing surface has a “U” shape.

16. An elevator for a medical device, comprising:
a first side wall; and
a second side wall,
wherein a proximal portion of the second side wall is substantially parallel to an entirety of the first side wall, and
wherein a distal portion of the second side wall curves toward the first side wall.

17. The elevator of claim 16, wherein a surface of the distal portion of the second side wall faces at least partially in a proximal direction.

18. The elevator of claim 6, wherein the elevator includes a guide surface extending from the first side wall to the second side wall, wherein the guide surface includes a first portion adjacent to the first side wall, a second portion adjacent to the second side wall, and a central portion between the first portion and the second portion, wherein the first portion and the central portion of the guide surface have open distal ends, and wherein the second portion of the guide surface has a closed distal end.

19. An elevator for a medical device, comprising:
a first side wall;
a second side wall; and
a guide surface for contacting an accessory device, wherein the guide surface extends between the first side wall and the second side wall, wherein the guide surface includes a first portion adjacent to the first side wall, a second portion adjacent to the second side wall, and a central portion between the first portion and the second portion,
wherein distal ends of the first portion and the central portion of the guide surface are open, and wherein the second side wall forms a closed distal end of the second portion.

20. The elevator of claim 19, wherein a surface of the second side wall faces at least partially in a proximal direction.
